(19) United States
(12) Patent Application Publication
(10) Pub. No.: US 2016/0159841 A1
(43) Pub. Date: Jun. 9, 2016

(54) LOW-ISOMERIZATION HYDROFORMYLATION OF OLEIC-ACID-ESTER-CONTAINING MIXTURES

(71) Applicants: Marcelo VILCHES, Santiago (CL); Susan LÜHR, Santiago (CL); Katrin Marie DYBALLA, Recklinghausen (DE); Robert FRANKE, Marl (DE); Armin BÖRNER, Rostock (DE); EVONIK DEGUSSA GMBH, Essen (DE)

(72) Inventors: Katrin Marie DYBALLA, Recklinghausen (DE); Robert FRANKE, Marl (DE); Armin BÖRNER, Rostock (DE); Susan LÜHR, Santiago (CL); Marcelo VILCHES, Santiago (CL)

(73) Assignee: EVONIK DEGUSSA GMBH, Essen (DE)

(21) Appl. No.: 14/906,730

(22) PCT Filed: Jul. 22, 2014
(86) PCT No.: PCT/EP2014/065747

§ 371 Date: Jan. 21, 2016

(30) Foreign Application Priority Data
Jul. 23, 2013 (DE) 10 2013 214 373.7
Jul. 23, 2013 (DE) 10 2013 214 382.6
Dec. 19, 2013 (EP) 13198274.6

(51) Int. Cl. C07F 9/6571; C07C 67/313; C07F 9/6584

(52) U.S. Cl. CPC C07F 9/657154; C07F 9/65848; C07C 67/313

(57) ABSTRACT

The invention relates to new ligands based on derivatives of phosphorous acid, to compositions that have said new ligands, and to the use thereof in a method for the low-isomerization hydroformylation of oleic-acid-ester-containing mixtures.

The invention relates to novel ligands based on derivatives of phosphorous acid, to compositions including these novel ligands and to the use thereof in a process for low-isomerization hydroformylation of mixtures containing oleic esters.

Frankel and co-workers in 1969 reported the hydroformylation of methyl oleate which is obtained from vegetable fatty acid esters such as soya oil, linseed oil and safflower oil. Under relatively severe conditions, 13.7 MPa (CO:H₂=1:1), 110° C., 5% Rh on CaCO₃ and 4.4% triphenylphosphine as ligand, the main product formed was the mixture of 9/10-formylstearates in 99% yield, as disclosed in J. Am. Oil Chem. Soc. 1969, 46, 133-138 and loc. cit. 1971, 48, 248-253. A disadvantage is that very severe reaction conditions were necessary here, for example pressures >10 MPa.

Some years later, Friedrich reported the hydroformylation of methyl oleate at much lower pressures of 1.38-6.21 MPa with triphenyl phosphite or triphenylphosphine as ligands, as disclosed in Ind. Eng. Chem. Res. Dev. 1978, 17, 205-207. The aldehyde was formed with up to a 97% yield. With triphenyl phosphite, there was an increase in the regioselectivity for formation of the 9/10-formyl product. A disadvantage is that very severe reaction conditions were necessary here, for example pressures >6 MPa.

Sterically demanding phosphites such as tris(4-tert-butyl-2-methylphenyl) phosphite were tested by van Leeuwen and co-workers in the hydroformylation of methyl oleate, as disclosed in J. Am. Oil Chem. Soc. 1997, 74, 223-228. Under mild conditions (2.0 MPa, CO:H₂=1:1, 80° C.), the aldehyde was obtained with 95% yield, assuming that pure methyl oleate was used. By contrast, the yield of 9/10-formyl product was very low with a technical grade methyl oleate (14% methyl linoleate).

Other studies on this topic were published by da Rosa and Gregório. In these studies, the hydroformylation of methyl oleate with different rhodium complexes such as ([RhH(CO)(PPh₃)₃], [RhCl₃×3H₂O], [RhCl(CO)(PPh₃)₃], [Rh(OMe)(COD)]₂ and [Rh(CO)₂(acac)]) and various ligands (triphenylphosphine and triphenyl phosphite) was examined. Up to 90%-95% yield of aldehyde was achieved at 100° C., 4.0 MPa (CO:H₂=2:1), as disclosed in J. Braz. Chem. Soc. 2005, 16, 1124-1129 and Quim. Nova 2012, 35, 1940-1944.

It is an object of the present invention to provide ligands which do not have the disadvantages described above in the prior art in the hydroformylation of mixtures containing oleic esters. This is specifically against the background of a low-isomerization hydroformylation and a high conversion of oleic esters to the derivatives a formylated preferentially in the C₈-C₁₃ position.

This object is achieved by ligands of the general formulae (I) or (II):

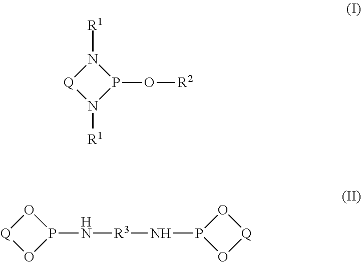

The present invention provides ligands of the general formula (I) or (II)

where Q is a substituted or unsubstituted aromatic radical;
where R′ is selected from alkyl, aryl, organosulfonyl radicals;
where R² is selected from a substituted or unsubstituted aromatic radical;
where R³ is a substituted or unsubstituted alkyl radical.

In a particular embodiment, Q is selected from substituted or unsubstituted 1,1′-biphenyl and 1,1′-binaphthyl radicals.

In one variant of this embodiment, R¹ is selected from a C₁-C₄-alkyl, alkylsulfonyl, arylsulfonyl radical, R² is an at least monosubstituted aromatic radical, R³ is a substituted or unsubstituted alkyl radical having at least 3 carbon atoms.

In a particularly preferred embodiment, the ligands of the formulae (I) and (II) are selected from L9-L14:

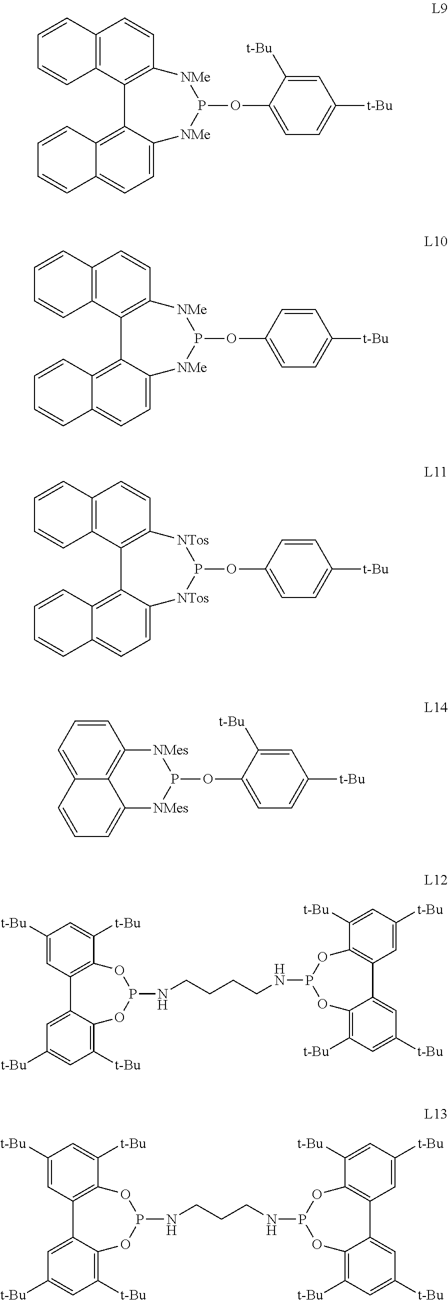

The present invention further provides transition metal compounds of the formula Me(acac)(CO)L with Me=transition metal of group 9 of the Periodic Table of the Elements, with acac=enolate anion of acetylacetone, where L=ligand selected from:

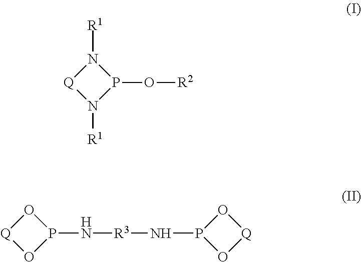

where Q is a divalent substituted or unsubstituted aromatic radical;
where R′ is selected from alkyl, aryl, organosulfonyl radicals;
where R² is selected from a substituted or unsubstituted aromatic radical;
where R³ is a substituted or unsubstituted alkyl radical,

In a particular embodiment, Q is selected from substituted or unsubstituted 1,1′-biphenyl and 1,1′-binaphthyl radicals.

In one variant of this embodiment, R¹ is selected from a C₁-C₄-alkyl, alkylsulfonyl, arylsulfonyl radical, R² is an at least monosubstituted aromatic radical, R³ is a substituted or unsubstituted alkyl radical having at least 3 carbon atoms.

In a particularly preferred embodiment, the compound of the formula Me(acac)(CO)L with Me=transition metal of group 9 of the Periodic Table of the Elements, with acac=enolate anion of acetylacetone, where L=ligand is selected from:

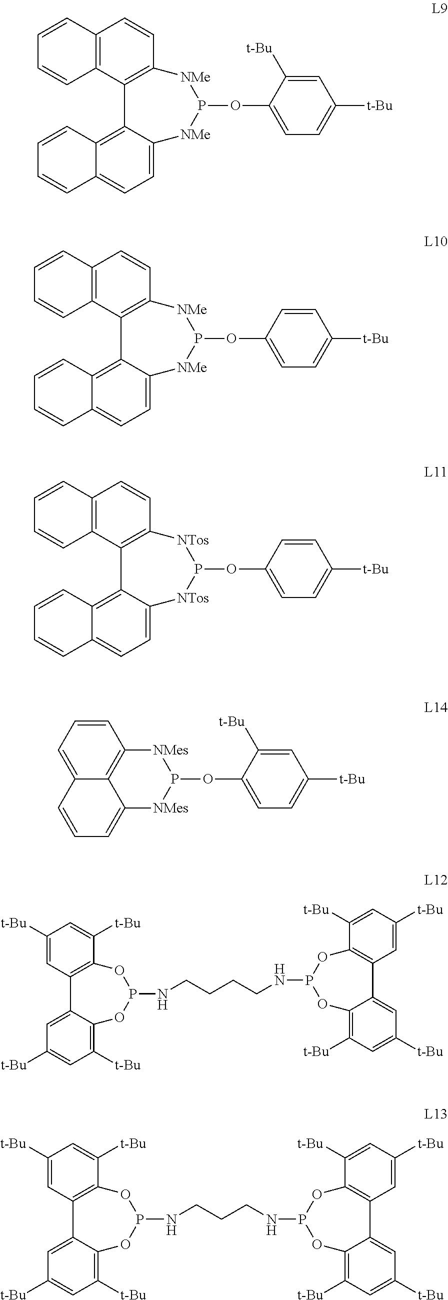

In one variant of this embodiment in which the transition metal is rhodium, the ligand/rhodium ratio is in the range from 100:1 to 1:1. Preferably, the ligand/rhodium ratio is in the range from 50:1 to 5:1, more preferably in the range from 30:1 to 20:1.

The transition metal is contacted with the inventive compounds of the formulae L9-L14 as a precursor in the form of its salts, for example the halides, carboxylates—e.g. acetates—or commercially available complexes, for example acetylacetonates, a carbonyls, cyclopolyenes—e.g. 1,5-cyclooctadiene—or else mixed forms thereof, for example Rh(acac)(CO)₂, Rh(acac)(COD) with COD=1,5-cyclooctadiene, and this reaction can be effected in a preceding reaction or else in the presence of a hydrogen- and carbon monoxide-containing gas mixture.

The present invention also provides compositions comprising:

2a) mixture containing oleic ester;
b) optionally at least one solvent;
c) at least one ligand of the formulae (I) or (II)
d) CO— and H₂-containing synthesis gas mixture;
e) at least one transition metal from group 9 of the Periodic Table of the Elements.

In one embodiment, the composition of the invention includes not only the oleic esters but also linoleic and linolenic esters.

In one variant of this embodiment, the esters are in the form of the methyl esters.

In one variant of this embodiment, ligands L1-L8 are used;

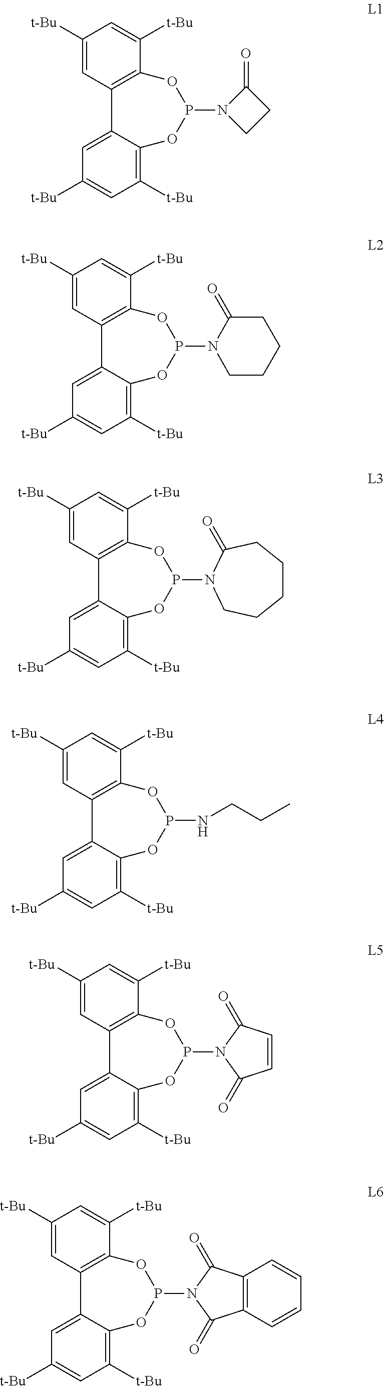 [SMILES: CC(C)(C)c1cc(C(C)(C)C)c2op(N3CCC3=O)oc3c(C(C)(C)C)cc(C(C)(C)C)cc3c2c1 ; CC(C)(C)c1cc(C(C)(C)C)c2op(N3CCCCC3=O)oc3c(C(C)(C)C)cc(C(C)(C)C)cc3c2c1 ; CC(C)(C)c1cc(C(C)(C)C)c2op(N3CCCCCC3=O)oc3c(C(C)(C)C)cc(C(C)(C)C)cc3c2c1 ; CCCNp1oc2c(C(C)(C)C)cc(C(C)(C)C)cc2c2cc(C(C)(C)C)cc(C(C)(C)C)c2o1 ; CC(C)(C)c1cc(C(C)(C)C)c2op(N3C(=O)C=CC3=O)oc3c(C(C)(C)C)cc(C(C)(C)C)cc3c2c1 ; CC(C)(C)c1cc(C(C)(C)C)c2op(N3C(=O)c4ccccc4C3=O)oc3c(C(C)(C)C)cc(C(C)(C)C)cc3c2c1]

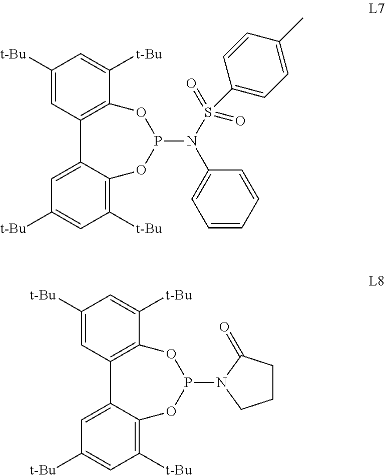 [SMILES: Cc1ccc(S(=O)(=O)N(c2ccccc2)p2oc3c(C(C)(C)C)cc(C(C)(C)C)cc3c3cc(C(C)(C)C)cc(C(C)(C)C)c3o2)cc1 ; CC(C)(C)c1cc(C(C)(C)C)c2op(N3CCCC3=O)oc3c(C(C)(C)C)cc(C(C)(C)C)cc3c2c1]

In one variant of this embodiment, ligands L9-L14 are used.

In one embodiment of the composition of the invention, the transition metal is rhodium.

In a further embodiment of the composition of the invention, the solvent is selected from a group comprising aromatic hydrocarbons, alcohols, ethers, carbonates.

In one variant of this embodiment, the aromatic hydrocarbon includes toluene or xylene, the alcohol methanol or ethanol, the ether tetrahydrofuran or MTBE, and the carbonate propylene carbonate.

The present invention further provides for the use of the compositions of the invention in a process for hydroformylating mixtures containing oleic esters.

The process of the invention for hydroformylating mixtures containing oleic esters using the compositions of the invention has synthesis gas pressures within a range of 1-6 MPa and reaction temperatures within a range of 60° C.-120° C.

In one embodiment of the process of the invention, the ratio of mixture containing oleic esters to transition metal is within a range from 180:1 to 910:1, the ratio of trivalent organic phosphorus compound to transition metal being within a range from 1:1 to 25:1.

In one variant of this embodiment, the ligands used are the following ligands L1-L8:

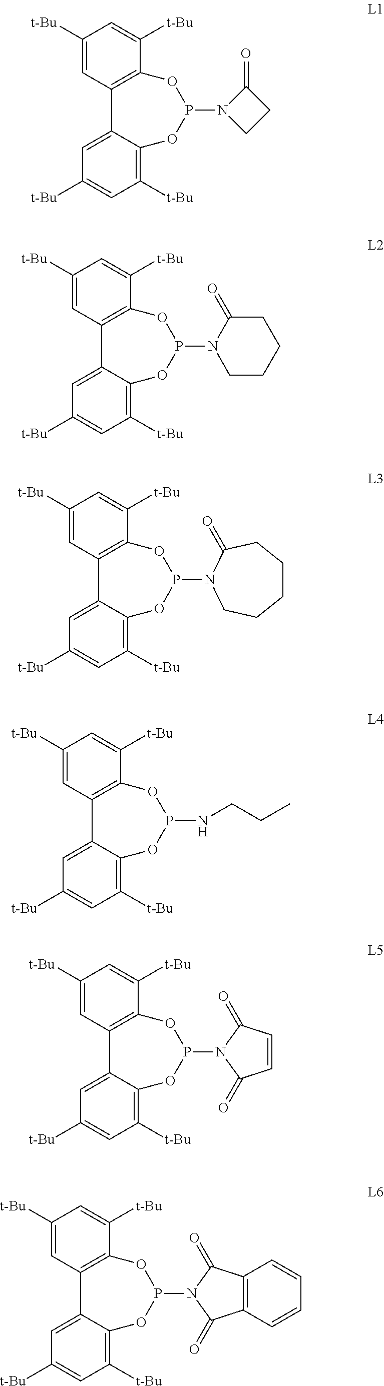 [SMILES: CC(C)(C)c1cc(C(C)(C)C)c2op(N3CCC3=O)oc3c(C(C)(C)C)cc(C(C)(C)C)cc3c2c1 ; CC(C)(C)c1cc(C(C)(C)C)c2op(N3CCCCC3=O)oc3c(C(C)(C)C)cc(C(C)(C)C)cc3c2c1 ; CC(C)(C)c1cc(C(C)(C)C)c2op(N3CCCCCC3=O)oc3c(C(C)(C)C)cc(C(C)(C)C)cc3c2c1 ; CCCNp1oc2c(C(C)(C)C)cc(C(C)(C)C)cc2c2cc(C(C)(C)C)cc(C(C)(C)C)c2o1 ; CC(C)(C)c1cc(C(C)(C)C)c2op(N3C(=O)C=CC3=O)oc3c(C(C)(C)C)cc(C(C)(C)C)cc3c2c1 ; CC(C)(C)c1cc(C(C)(C)C)c2op(N3C(=O)c4ccccc4C3=O)oc3c(C(C)(C)C)cc(C(C)(C)C)cc3c2c1]

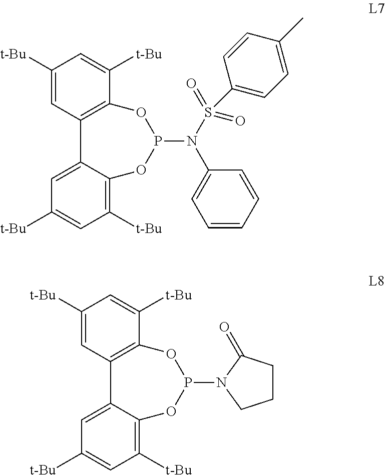 [SMILES: Cc1ccc(S(=O)(=O)N(c2ccccc2)p2oc3c(C(C)(C)C)cc(C(C)(C)C)cc3c3cc(C(C)(C)C)cc(C(C)(C)C)c3o2)cc1 ; CC(C)(C)c1cc(C(C)(C)C)c2op(N3CCCC3=O)oc3c(C(C)(C)C)cc(C(C)(C)C)cc3c2c1]

In one variant of this embodiment, the ligands used are ligands L9-L14.

The inventive compositions and the use thereof are described hereinafter by way of example, without any intention that the invention be restricted to these illustrative embodiments. When ranges, general formulae or compound classes are specified hereinafter, these shall include not just the corresponding ranges or groups of compounds that are explicitly mentioned but also all sub-ranges and sub-groups of compounds which can be obtained by removing individual values (ranges) or compounds. When documents are cited in the context of the present description, the contents thereof, particularly with regard to the subject matter that forms the context in which the document has been cited, are considered in their entirety to form part of the disclosure content of the present invention. Unless stated otherwise, percentages are figures in percent by weight. When average values are reported hereinbelow, the values in question are weight averages, unless stated otherwise. When parameters which have been determined by measurement are reported hereinafter, they have been determined at a temperature of 25° C. and a pressure of 101.325 Pa, unless stated otherwise.

The term “inert” in the context of the present invention is understood to mean the property of substances, components or mixtures of causing no adverse effects or effects contrary to the intended course of the reaction.

### EXAMPLES

### General Procedures

All the preparations which follow were carried out under protective gas using standard Schlenk techniques. The solvents were dried over suitable desiccants before use (Purification of Laboratory Chemicals, W. L. F. Armarego (Author), Christina Chai (Author), Butterworth Heinemann (Elsevier), 6th edition, Oxford 2009).

Phosphorus trichloride (Aldrich) was distilled under argon before use. All preparative operations were effected in baked-out vessels. The products were characterized by means of NMR spectroscopy. Chemical shifts are reported in ppm. The ³¹P NMR signals were referenced as follows: SR_{31P}═SR_{1H}*(BF_{31P}/BF_{1H})═SR_{1H}*0.4048. (Robin K. Harris, Edwin D. Becker, Sonia M. Cabral de Menezes, Robin Goodfellow, and Pierre Granger, Pure Appl. Chem., 2001, 73, 1795-1818; Robin K. Harris, Edwin D. Becker, Sonia M. Cabral de Menezes, Pierre Granger, Roy E. Hoffman and Kurt W. Zilm, Pure Appl. Chem., 2008, 80, 59-84).

The recording of nuclear resonance spectra was effected on Bruker Avance 300 or Bruker Avance 400, gas chromatography analysis on Agilent GC 7890A, elemental analysis on Leco TruSpec CHNS and Varian ICP-OES 715, and ESI-TOF mass spectrometry on Thermo Electron Finnigan MAT 95-XP and Agilent 6890 N/5973 instruments.

### Reaction Scheme: Hydroformylation of Methyl Oleate

Under hydroformylation conditions, the reaction with methyl oleate (MO) and mixtures thereof as substrate can give not only the desired methyl C8-C13-formylstearate (MFS) but also the isomerized olefin (methyl elaidate=ME) and the hydrogenation product (methyl stearate=MS).

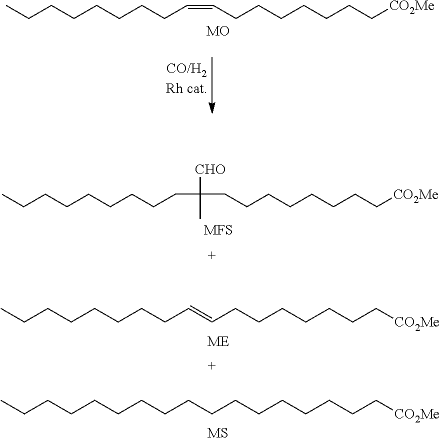 [SMILES: CCCCCCCC/C=C\CCCCCCCC(=O)OC ; CCCCCCCCCC(C)(C=O)CCCCCCCCC(=O)OC ; CCCCCCCC/C=C/CCCCCCCC(=O)OC ; CCCCCCCCCCCCCCCCCC(=O)OC]

### Comparative Example

### Low-Isomerization Hydroformylation of Methyl Oleate

[Rh(acac)(CO)₂](2.1 mg, 4.0 μmol) was weighed out in a Schlenk vessel under argon and dissolved in 2 mL of toluene. 1 mL of this solution together with methyl oleate (2.0 mmol, 0.593 g, >99% purity), triphenylphosphine (5.3 μmol) and 9 mL of toluene were introduced into a 25 mL autoclave. The autoclave was purged three times with nitrogen (1.0 MPa) and once with synthesis gas (CO:H₂=1:1, 1.0 MPa) and then heated to 80° C. and the pressure was adjusted to 5.0 MPa. After a reaction time of 24 h, the autoclave is cooled down. Subsequently, the pressure was released at room temperature and the system was purged twice with nitrogen. A sample was taken from the autoclave for the GC-MS analysis. The solvent was evaporated and the yellow oil was analyzed by NMR (¹H, ¹³C, DEPT) (99% conversion of MO, 99% MFS).

### Inventive Examples

### Hydroformylation of Methyl Oleate with Ligands L1-L14

[Rh(acac)(CO)₂](1.4 mg, 5.43 μmol) was weighed out in a Schlenk vessel under argon and dissolved in 5 mL of toluene. 1 g of this solution together with methyl oleate (1.0 mmol, 0.296 g, >99% purity), ligand (27.5 μmol), octadecane (0.1 g) as internal standard and 9 mL of toluene were introduced into a 25 mL autoclave. The autoclave was purged three times with nitrogen (1.0 MPa) and once with synthesis gas (CO:H₂=1:1, 1.0 MPa) and then heated to 80° C. and the pressure was adjusted to 20 bar. After a reaction time of 6 h, the autoclave was cooled down. At room temperature, the pressure was released and the system was purged twice with nitrogen. A sample was taken from the autoclave for the GC-MS analysis. The solvent was evaporated and the yellow oil was analyzed by NMR (1H, 13C, DEPT).

### Analysis for Determination of Regioselectivity

Characterization and calibration of the product (MFS) was accomplished using an isomerization-free hydroformylation with triphenylphosphine according to the method of Vogl et al., PhD thesis, Rostock, 2009, as described hereinafter.

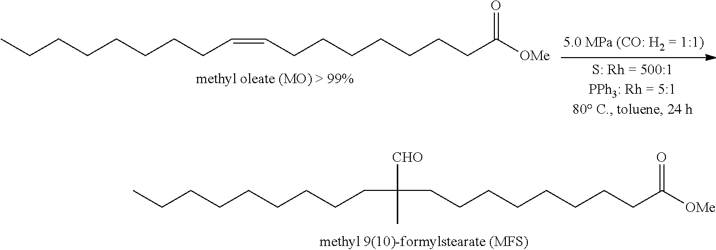 [SMILES: CCCCCCCC/C=C\CCCCCCCC(=O)OC ; CCCCCCCCCC(C)(C=O)CCCCCCCCC(=O)OC]

To purify the hydroformylation products of methyl oleate, the reaction mixture was distilled in a Kugelrohr distillation apparatus (1.5×10⁻¹ mbar/180° C.). Pure methyl formylstearate was used for the calibration, employing octadecane as internal standard.

In attempts to purify the product by means of column chromatography (cyclohexane:ethyl acetate), the formyl product decomposed on the column.

In order to determine the exact position of the aldehyde group, the products of the hydroformylation were analyzed by GC-MS. The fact that the aldehydes are very air-sensitive is known from the prior art: Frankel et al. in J. Am. Oil Chem. Soc. 1969, 46, 133-138 and loc. cit. 1971, 48, 248-253. The aldehydes were oxidized to the corresponding acids and then converted to the methyl esters. The latter were then analyzed by GC-MS. The following scheme summarizes the steps:

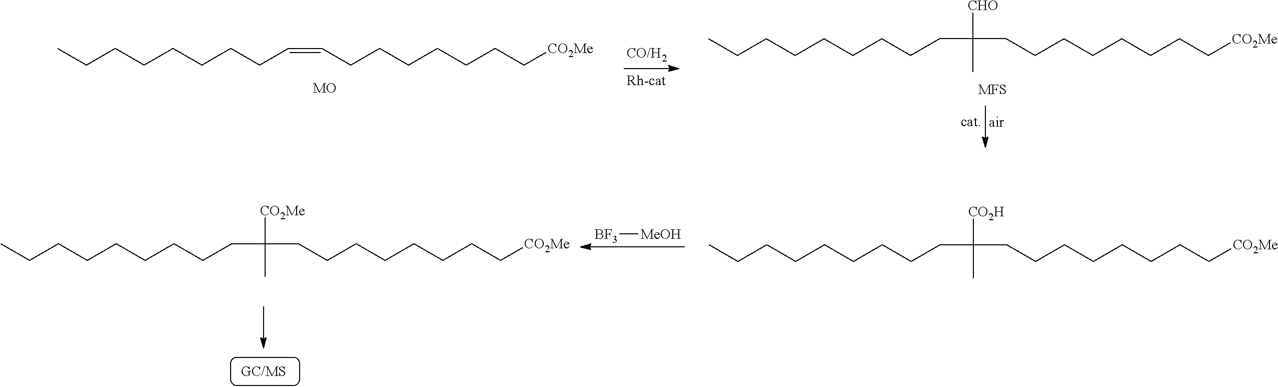

In order to determine the exact position of the aldehyde group, the products of the hydroformylation were converted to the corresponding methyl esters and analyzed by GC-MS. The branched formyl products which originate from hydroformylation between carbon atoms 3 and 17 are characterized by the fragment CH3(CH2)ₙCHC(O+.H)OCH3, and for the linear product (18-MFS) by the fragment CHC(O+.H)OCH3.

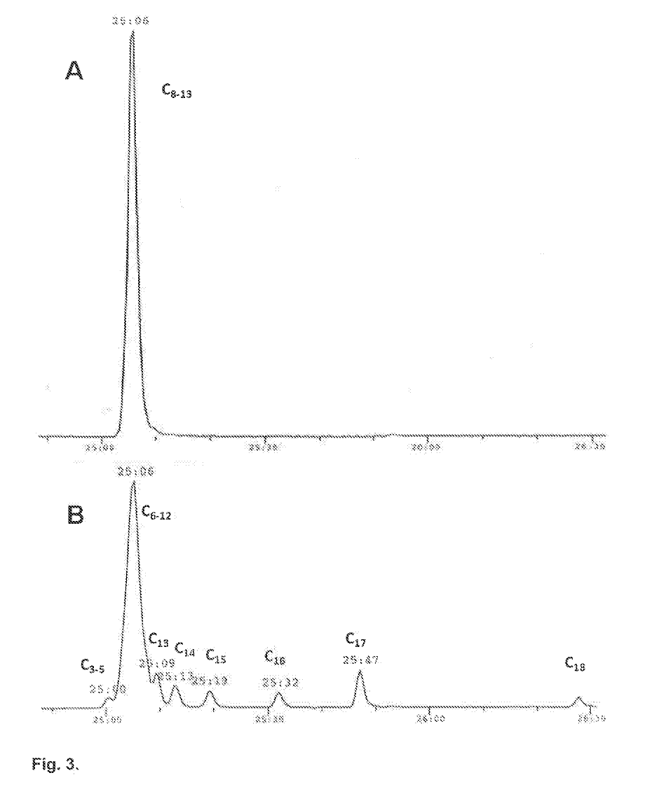

The gas chromatography-resolved mass spectrum A in FIG. 3 which results from hydroformylation at 20 bar and 80° C. one observes a single large peak characterized mainly by the mixture of 9- (fragment 200 m/z) and 10-MFS (fragment 186 m/z). In addition, a smaller amount of 8- (214 m/z), 11- (172 m/z), 12- (158 m/z) and 13-MFS (144 m/z) is found. These regioisomers are found at the start and end of the gas chromatogram; separation of the main isomers is unsuccessful.

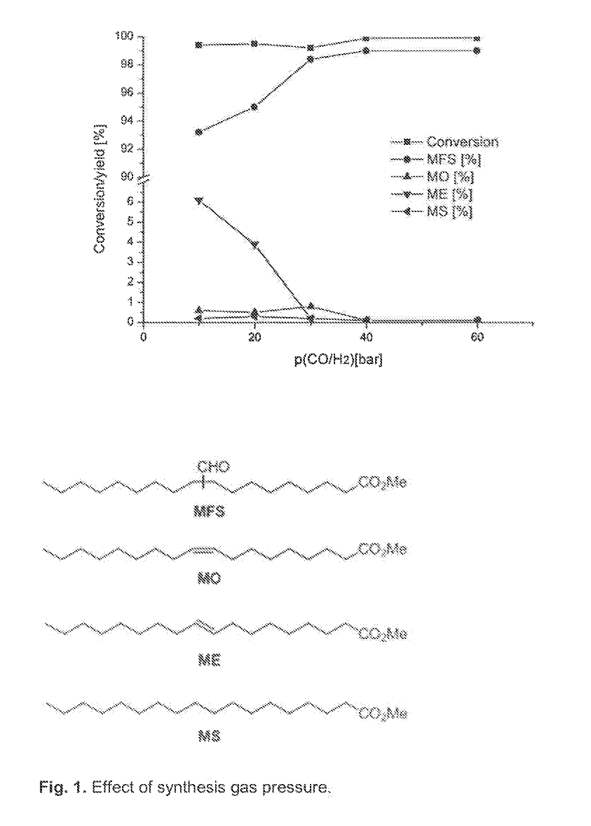

The gas chromatography-resolved mass spectrum B in FIG. 3 which was recorded after the workup of the hydroformylation at 20 bar at 120° C. shows a lower degree of regioselectivity, which is demonstrated by the occurrence of additional peaks. The first individual peak corresponds to the mixture of 3- (284 m/z), 4- (270 m/z) and 5-MFS (256 m/z). The main peak is characterized, as already shown in FIG. 1A, by a mixture of 6-to 12-MFS (242 m/z and 228 m/z for 6- and 7-MFS). The products 13-MFS (144 m/z), 14- (130 m/z), 15- (116 m/z), 16- (102 m/z), 17- (88 m/z), and 18-MFS (74 m/z) are well-separated and can thus be observed as individual peaks.

Mass spectrum of the isomeric methyl 13- to 18-methoxycarboxystearates;

Methyl 13-methoxycarboxystearate: MS (EI, 70 eV): m/z: 325, 297, 254, 222, 144, 98, 87, 55, 41.
Methyl 14-methoxycarboxystearate: MS (EI, 70 eV): m/z: 324, 297, 268, 227, 154, 130, a 98, 87, 55, 41.
Methyl 15-methoxycarboxystearate: MS (EI, 70 eV): m/z: 325, 297, 241, 196, 140, 116, 98, 87, 55, 41.
Methyl 16-methoxycarboxystearate: MS (EI, 70 eV): m/z: 324, 297, 255, 223, 182, 126, 102, 87, 55, 41.
Methyl 17-methoxycarboxystearate: MS (EI, 70 eV): m/z: 324, 297, 269, 237, 210, 112, 98, 88, 55, 41.
Methyl 18-methoxycarboxystearates: MS (EI, 70 eV): m/z: 325, 283, 251, 210, 112, 98, 74, 55, 41.

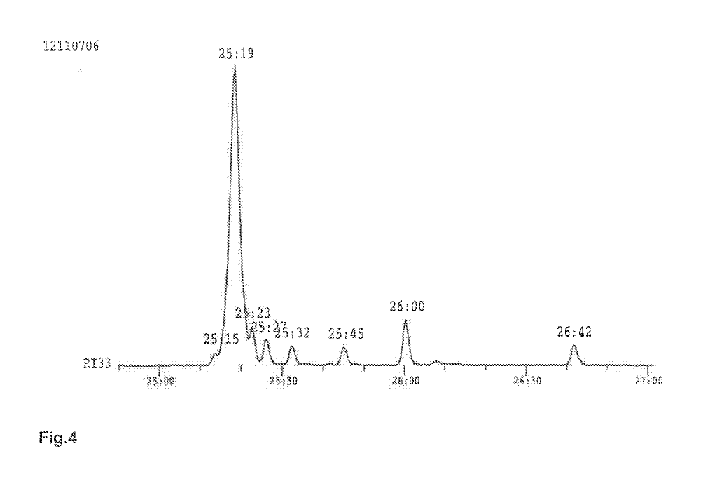

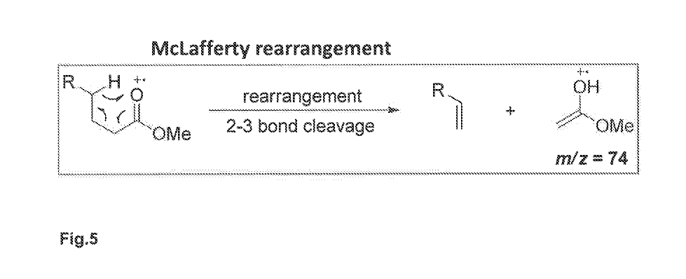

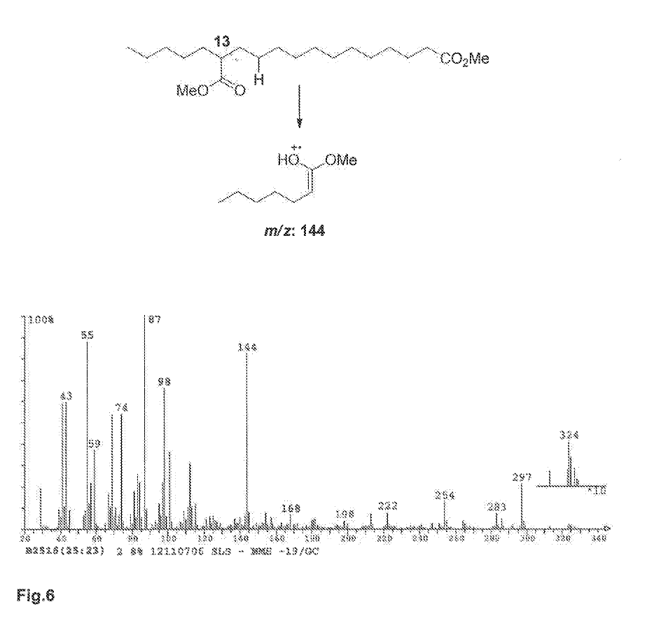

The assignment of the respective mass spectra and the quantification of the respective formylstearate derivatives prepared that has been derived therefrom were effected on the basis of the McLafferty rearrangement known to those skilled in the art. The results are disclosed in FIGS. 4, 5 and 6.

Mixture of methyl 9- and 10-formyloctadecanoate:

1H NMR (300 MHz, CDCl3): δ 0.91 (t, 3H, J=7.0 Hz, CH3), 1.25-1.38 (m, 22H, CH2), 1.40-1.55 (m, 2H, CH2CH2CO2CH3), 1.57-1.73 (m, 3H, CH2CH(CHO)CH2), 2.20-2.30 (m, 1H, CHCHO), 2.34 (t, 2H, J=7.6 Hz, CH2CO2CH3), 3.70 (s, 3H, CO2CH3), 9.59 (d, 1H, J=3.2 Hz, CHO).

13C NMR (75 MHz, CDCl3): δ 14.1 (s, CH3), 22.7-32.0 (m, CH2), 34.1 (s, CH2CO2CH3), 34.1 (s, CH2CO2CH3), 51.5 (s, CO2CH3), 52.0 (s, CHCHO), 174.3 (s, CO2CH3), 174.3 (s, CO2CH3), 205.7 (s, CHO), 205.7 (s, CHO).

MS (EI, 70 eV): m/z (I, %): 326 (<0.83), 298 (46.2), 269 (5.3), 255 (28.3), 241 (4.9), 213 (6.4), 199 (17.6), 171 (27.1), 139 (34.2), 125 (32.1), 111 (22.9), 98 (47.01), 87 (95.9), 74 (77.1), 69 (58.2), 55 (100), 43 (75.7).

### Oxidation of Methyl Formyloctadecanoate

About 100 mg of crude product were left to stand under air for 48 hours. The white solid obtained was analyzed by NMR (1H, 13C, DEPT); quantitative conversion, 99% yield.

1H NMR (300 MHz, CDCl3): δ 0.87 (t, 3H, J=6.8 Hz, CH3), 1.15-1.37 (m, 22H, CH2), 1.39-1.51 (m, 2H, CH2), 1.53-1.71 (m, 4H, CH2), 2.29 (t, 2H, J=7.6 Hz, CH2CO2CH3), 2.35 (m, 1H, CHCO2H), 3.66 (s, 3H, CO2CH3).

13C NMR (75 MHz, CDCl3): δ 14.3 (s, CH3), 22.8-32.3 (m, CH2), 34.2 (s, CH2CO2CH3), 34.2 (s, CH2CO2CH3), 45.6 (s, CHCO2H), 51.6 (s, CO2CH3), 174.5 (s, CO2CH3), 174.5 (s, CO2CH3), 182.4 (s, CO2H).

### Esterification of Methyl Carboxyoctadecanoate

About 30 g of the free acid were dissolved in 1.0 mL of toluene in a test tube. Subsequently, 2.0 mL of a solution of boron trifluoride in methanol (10%) and three drops of 2,2-dimethoxypropane as dehydrating agent were added. The reaction mixture was heated to 60° C. in a water bath for 25 minutes. After the addition of 1.0 mL of distilled water, the phases were separated. The organic phase was dried with Na₂SO₄ and then filtered; quantitative conversion, 99% yield. The solution was analyzed by GC-MS.

MS (EI, 70 eV): m/z (l, %): 324 (<1.40), 297 (28.9), 283 (8.0), 264 (6.3), 244 (8.6), 230 (10.7), 212 (25.4), 200 (46.7), 186 (45.7), 157 (25.1), 143 (35.94), 112 (20.2), 98 (93.7), 87 (100), 74 (42.3), 69 (42.6), 55 (80.3), 43 (47.7).

### Use of L1 as Ligand

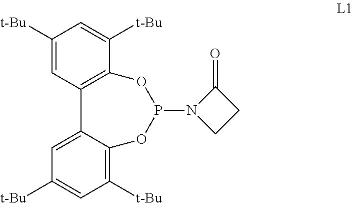 [SMILES: CC(C)(C)c1cc(C(C)(C)C)c2op(N3CCC3=O)oc3c(C(C)(C)C)cc(C(C)(C)C)cc3c2c1]

### Effect of Synthesis Gas Pressure

The hydroformylation of MO was tested at a reaction temperature of 80° C. and five different pressures (1.0, 2.0, 3.0, 4.0 and 6.0 MPa of synthesis gas CO:H₂=1:1) (FIG. 1).

TABLE 1
Hydroformylation of MO at different pressures with L1 as ligand.^{a}

| | Conversion | MO | MFS | ME | MS | |
|---|---|---|---|---|---|---|
| p [MPa] | [%]^{b} | [%]^{c} | [%]^{c} | [%]^{c} | [%]^{c} | Regioselectivity^{d} |
| 1.0 | 99.4 | 0.6 | 93.2 | 6.1 | 0.2 | C5-C17 |
| 2.0 | 99.5 | 0.5 | 95.0 | 3.9 | 0.3 | C8-C13 |
| 3.0 | 99.2 | 0.8 | 98.4 | 0.2 | 0.2 | C8-C13 |
| 4.0 | 99.9 | — | 99.0 | — | — | C8-C13 |
| 6.0 | 99.9 | — | 99.0 | — | — | C8-C12 |

^{a}Reaction conditions: 1 mmol of substrate (S), S:Rh = 910:1, P:Rh = 25:1, 80° C., 10 mL of toluene.
^{b}Conversions were determined by GC (100% GC yield of MO).
^{c}Determined by GC.
^{d}Cn, n = carbon atoms where the hydroformylation took place.

Table 1 shows clearly that an increase in pressure leads to a higher yield of MFS, but the increase in this case is very steep from 2.0 MPa upward (from 95% at 2.0 MPa to >98% at 3.0 MPa). At higher gas pressures (>2.0 MPa), chemoselectivity improves in the hydroformylation direction. In all cases, the formation of the hydrogenation product MS is very low (0.1%-0.3%).

Regioselectivity is likewise dependent on synthesis gas pressure. At higher pressures, regioselectivity increases (table 1).

### Effect of Reaction Temperature

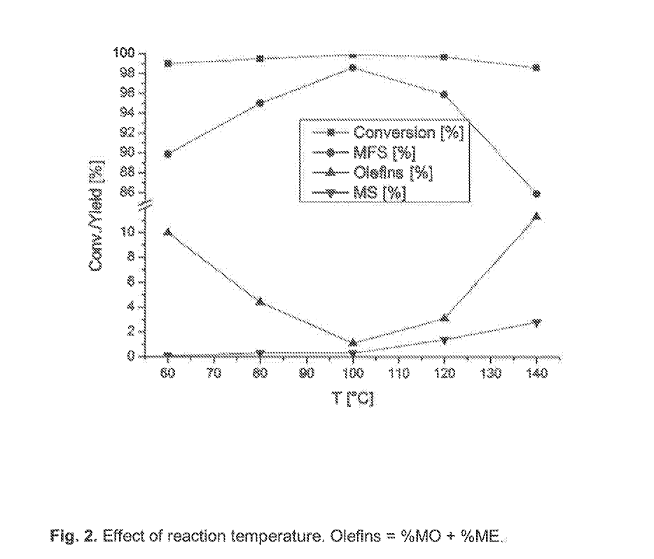

The reaction was conducted at 2.0 MPa at five different temperatures (60, 80, 100, 120, 140° C.) (FIG. 2).

TABLE 2
Hydroformylation of MO at different temperatures with L1 as ligand.^{a}

| | Conversion | MO | MFS | ME | MS | |
|---|---|---|---|---|---|---|
| T [° C.] | [%]^{b} | [%]^{c} | [%]^{c} | [%]^{c} | [%]^{c} | Regioselectivity^{d} |
| 60 | 99.0 | 1.0 | 89.9 | 9.0 | 0.1 | C9-C12 |
| 80 | 99.5 | 0.5 | 95.0 | 3.9 | 0.3 | C8-C13 |
| 100 | 99.9 | 0.1 | 98.6 | 1.0 | 0.3 | C3-C18 |
| 120 | 99.7 | 0.3 | 95.9 | 2.8 | 1.4 | C3-C18 |
| 140 | 98.6 | 1.4 | 85.9 | 9.9 | 2.8 | C3-C18 |

^{a}Reaction conditions: 1 mmol of substrate (S), S:Rh = 910:1, P:Rh = 25:1, 2.0 MPa of synthesis gas (CO:H₂ = 1:1), 10 mL of toluene.
^{b}Conversions were determined by GC (100% GC yield of MO).
^{c}Determined by GC.
^{d}Cn, n = carbon atoms where the hydroformylation took place.

Chemoselectivity increased at first even at higher temperatures. With a maximum of 100° C., it decreased again at 120 and 140° C. It appears that three reactions compete with one another at temperatures higher than 120° C.: 1.) hydroformylation to give MFS 2.) decarbonylation to give the corresponding olefin and 3.) hydrogenation.

At 100, 120 and 140° C., there is a distinct decrease in regioselectivity (table 2).

### Effect of Solvent

When the hydroformylation of methyl oleate has been conducted in methanol as solvent, conversion (39.1%) and yield of MFS (25.7%) decrease. Even though the best regioselectivity (only the C9-C10 mixture) is found with this solvent, products unidentifiable even by mass spectroscopy occur in the gas chromatogram.

### Variation of Ligands

Under optimized conditions (2.0 MPa, 80° C., toluene), phosphoramidites were tested as ligands in the hydroformylation of methyl oleate.

For comparison, the results were compared with commercially available triphenylphosphine. 12 monodentate and 2 bidentate ligands were examined:

TABLE 3
Hydroformylation of MO with inventive ligands
L1-L14^{a} compared to PPh₃

| | Conversion | MO | MFS | ME | MS |
|---|---|---|---|---|---|
| Ligand | [%]^{b} | [%]^{c} | [%]^{c} | [%]^{c} | [%]^{c} |
| PPh₃ | 59.0 | 41.0 | 51.8 | 7.1 | 0.1 |
| L1^{d} | 99.5 | 0.5 | 95.0 | 3.9 | 0.3 |
| L2 | 88.7 | 11.3 | 32.2 | 56.2 | 0.3 |
| L3 | 98.7 | 1.3 | 89.1 | 9.3 | 0.3 |
| L4 | 92.9 | 7.1 | 43.8 | 49.0 | 0.1 |
| L5 | 46.2 | 53.8 | 0.7 | 45.2 | 0.3 |
| L6 | 91.6 | 8.4 | 39.9 | 51.5 | 0.2 |
| L7 | 95.8 | 4.2 | 54.7 | 28.9 | 1.1 |
| L8 | 91.5 | 8.5 | 32.9 | 58.1 | 0.5 |
| L9 | 32.8 | 67.2 | 25.5 | 7.3 | 0 |
| L10 | 92.3 | 7.7 | 27.7 | 64.3 | 0.3 |
| L11 | 94.4 | 5.6 | 39.5 | 54.3 | 0.6 |
| L12 | 68.6 | 31.4 | 51.5 | 17.0 | 0.1 |
| L13 | 1.6 | 98.3 | 1.5 | 0.1 | 0.1 |
| L14 | 93.5 | 6.5 | 32.3 | 60.1 | 1.2 |
| L4^{a} | 91.4 | 8.6 | 38.2 | 52.8 | 0.4 |

^{a}Reaction conditions: 1.0 mmol of substrate (S), S:Rh = 910:1. P:Rh = 25:1, 20 bar of synthesis gas (CO:H₂ = 1:1), 80° C., 10 mL of toluene (The reaction mixture was stirred at 80° C. for 6 h and then stirring was continued at room temperature under syngas pressure until processing.)
^{b}Conversions were determined by GC (100% GC yield of MO).
^{c}Determined by GC.
^{d}Average between two reactions.
^{e}After 6 h, the reaction was worked up and the sample was analyzed by GC-MS.

The best results were achieved for the 4-membered and 7-membered lactam rings L1 and L3 with a yield of 92.9% and 89.1% of MFS. The triphenylphosphine ligand which is mentioned in the prior art and is commercially available features an inadequate conversion of only just over 60% under these distinctly milder reaction conditions compared to the prior art.

As a conclusion, it is found that a hydroformylation of mixtures containing oleic esters with conversions >85% to give the derivatives formylated in the C8-C13 position using the ligands L1 and L3 is obtainable. The hydroformylation is conducted preferentially internally, and hence with low isomerization.

1. A ligand selected from the group comprising:

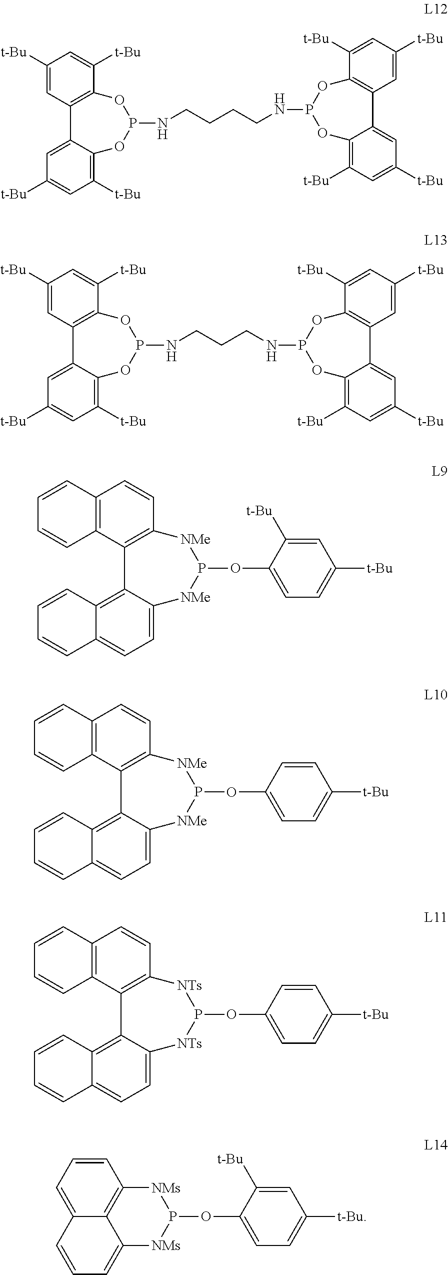

2. A transition metal compound of the formula Me(acac)(CO)L with Me=group 9 transition metal, where L=ligand as claimed in claim 1.

3. The transition metal compound as claimed in claim 2, characterized in that it includes rhodium.

4. A composition comprising:
a) mixture containing oleic esters;
b) optionally at least one solvent;
c) at least one ligand as claimed in claim 1 or one of ligands L1 to L8: 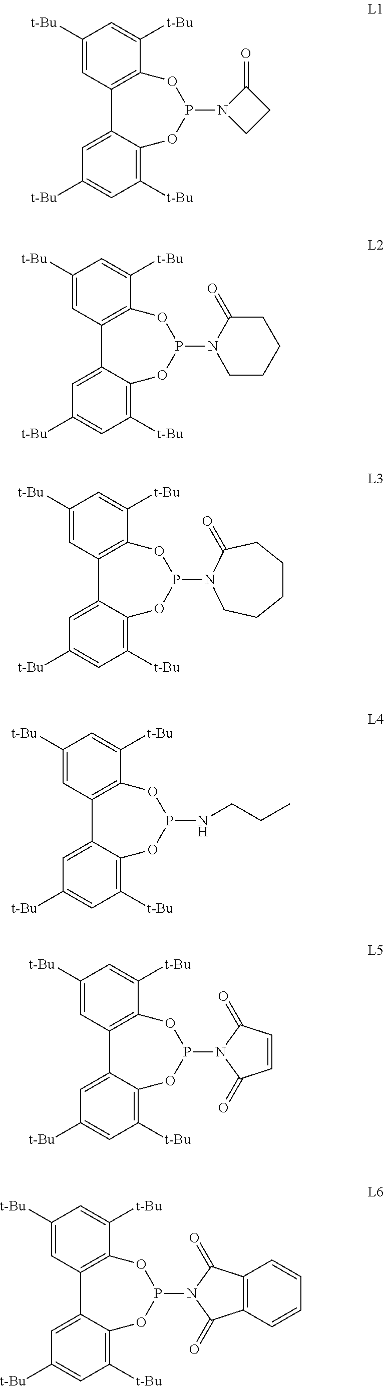 [SMILES: CC(C)(C)c1cc(C(C)(C)C)c2op(N3CCC3=O)oc3c(C(C)(C)C)cc(C(C)(C)C)cc3c2c1 ; CC(C)(C)c1cc(C(C)(C)C)c2op(N3CCCCC3=O)oc3c(C(C)(C)C)cc(C(C)(C)C)cc3c2c1 ; CC(C)(C)c1cc(C(C)(C)C)c2op(N3CCCCCC3=O)oc3c(C(C)(C)C)cc(C(C)(C)C)cc3c2c1 ; CCCNp1oc2c(C(C)(C)C)cc(C(C)(C)C)cc2c2cc(C(C)(C)C)cc(C(C)(C)C)c2o1 ; CC(C)(C)c1cc(C(C)(C)C)c2op(N3C(=O)C=CC3=O)oc3c(C(C)(C)C)cc(C(C)(C)C)cc3c2c1 ; CC(C)(C)c1cc(C(C)(C)C)c2op(N3C(=O)c4ccccc4C3=O)oc3c(C(C)(C)C)cc(C(C)(C)C)cc3c2c1] 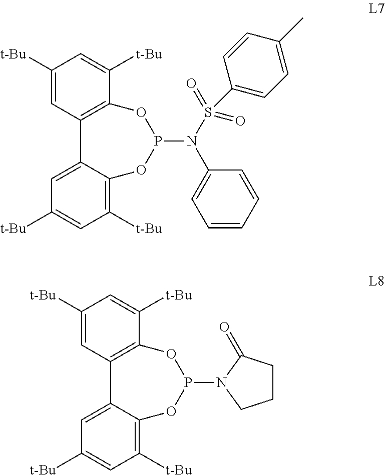 [SMILES: Cc1ccc(S(=O)(=O)N(c2ccccc2)p2oc3c(C(C)(C)C)cc(C(C)(C)C)cc3c3cc(C(C)(C)C)cc(C(C)(C)C)c3o2)cc1 ; CC(C)(C)c1cc(C(C)(C)C)c2op(N3CCCC3=O)oc3c(C(C)(C)C)cc(C(C)(C)C)cc3c2c1]
d) CO— and H₂-containing synthesis gas mixture;
e) at least one transition metal from group 9 of the Periodic Table of the Elements.

5. The composition as claimed in claim 4,
wherein not only the oleic ester but also linoleic and linolenic esters are present.

6. The composition as claimed in claim 5,
wherein the esters include methyl esters.

7. The composition as claimed in claim 4, including one of the following ligands:

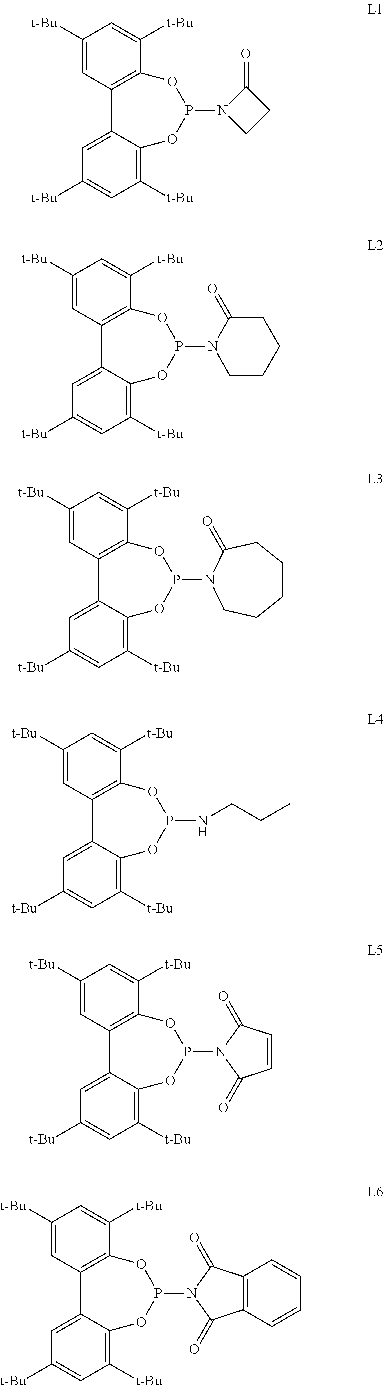 [SMILES: CC(C)(C)c1cc(C(C)(C)C)c2op(N3CCC3=O)oc3c(C(C)(C)C)cc(C(C)(C)C)cc3c2c1 ; CC(C)(C)c1cc(C(C)(C)C)c2op(N3CCCCC3=O)oc3c(C(C)(C)C)cc(C(C)(C)C)cc3c2c1 ; CC(C)(C)c1cc(C(C)(C)C)c2op(N3CCCCCC3=O)oc3c(C(C)(C)C)cc(C(C)(C)C)cc3c2c1 ; CCCNp1oc2c(C(C)(C)C)cc(C(C)(C)C)cc2c2cc(C(C)(C)C)cc(C(C)(C)C)c2o1 ; CC(C)(C)c1cc(C(C)(C)C)c2op(N3C(=O)C=CC3=O)oc3c(C(C)(C)C)cc(C(C)(C)C)cc3c2c1 ; CC(C)(C)c1cc(C(C)(C)C)c2op(N3C(=O)c4ccccc4C3=O)oc3c(C(C)(C)C)cc(C(C)(C)C)cc3c2c1]

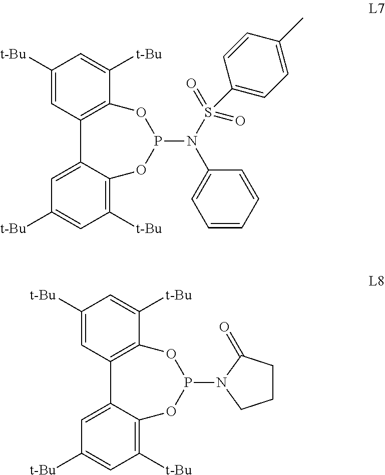 [SMILES: Cc1ccc(S(=O)(=O)N(c2ccccc2)p2oc3c(C(C)(C)C)cc(C(C)(C)C)cc3c3cc(C(C)(C)C)cc(C(C)(C)C)c3o2)cc1 ; CC(C)(C)c1cc(C(C)(C)C)c2op(N3CCCC3=O)oc3c(C(C)(C)C)cc(C(C)(C)C)cc3c2c1]

8. The composition as claimed in claim 4,
wherein the transition metal is rhodium.

9. The composition as claimed in claim 4,
wherein the solvent is selected from the group comprising aromatic hydrocarbons, alcohols, ethers, carbonates.

10. The composition as claimed in claim 9,
wherein the aromatic hydrocarbon includes toluene or xylene, the alcohol methanol or ethanol, the ether tetrahydrofuran or MTBE, and the carbonate propylene carbonate.

11. A process for hydroformylating mixtures containing oleic esters comprising introducing the composition of claim 4.

12. A process for hydroformylating mixtures containing oleic esters using a composition as claimed in claim 4, wherein the synthesis gas pressure is within a range of 1-6 MPa and the reaction temperature within a range of 60° C.-120° C.

13. The process as claimed in claim 12, wherein the ratio of mixture containing oleic esters to transition metal is within a range from 180:1 to 910:1, the ratio of trivalent organic phosphorus compound to transition metal being within a range from 1:1 to 25:1.
